(19) United States
(12) Patent Application Publication
(10) Pub. No.: US 2013/0190541 A1
(43) Pub. Date: Jul. 25, 2013

(54) CATALYST SYSTEM, COMPRISING CATALYST PELLETS AND DILUENT BEADS WITH PREDEFINED DIMENSIONS AND PHYSICOCHEMICAL PROPERTIES

(71) Applicants: Lorette Du Preez, Woluwe (BE); Stephen Kevin Lee, London (GB); Andrea Marsella, Paese (TV) (IT); Sandro Vidotto, Pordenone (PN) (IT)

(72) Inventors: Lorette Du Preez, Woluwe (BE); Stephen Kevin Lee, London (GB); Andrea Marsella, Paese (TV) (IT); Sandro Vidotto, Pordenone (PN) (IT)

(73) Assignee: INEOUS EUROPE AG, Rolle (CH)

(21) Appl. No.: 13/878,891

(22) PCT Filed: Jul. 1, 2011
(86) PCT No.: PCT/EP11/61092

§ 371 Date: Apr. 11, 2013

(30) Foreign Application Priority Data
Oct. 21, 2010 (CH) 1017804.4

(51) Int. Cl. B01J 35/02; C07C 17/23; C07C 17/10

(52) U.S. Cl. CPC B01J 35/026; C07C 17/10; C07C 17/23
USPC 570/220; 570/230; 422/129; 252/71

(57) ABSTRACT

A catalyst system for use in oxychlorination, the catalyst system comprising catalyst pellets comprising a catalyst carried on a substrate the pellets having length x, breadth y and depth z, intrinsic density P and bulk density p and diluent beads having length x±25%, breadth y±25% and depth z±25%, intrinsic density≧P+25% and a bulk density p ±25%.

This invention relates to catalyst system for oxychlorination. More especially but not exclusively the invention relates to catalyst systems for fixed bed oxychlorination.

Typical oxychlorination processes involve the conversion of ethylene, C₂H₄, into 1,2-dichloroethane, ClCH₂CH₂Cl. 1,2 dichloroethane is known by a variety of other names including ethylene dichloride and EDC. It is a useful precursor for a range of industrial chemicals including vinyl chloride and ethylene diamine. It is also a useful solvent. Vinyl chloride is prepared by dehydrohalogenation of 1,2-dichloroethane for example by heating at elevated pressure.

The reaction for the production of 1,2-dichloroethane from ethylene is of formula

C₂H₄+2HCl+0.5O₂→ClCH₂CH₂Cl+H₂O

and is typically catalysed by copper II supported on a substrate such as alumina. Typically the catalyst is presented as pellets. Pellets of a wide range of shapes and dimensions have been proposed. In general pellets comprise an alumina base carrying a copper containing catalyst.

The catalysts in pellets are loaded in the tubes of the fixed bed reactors in a loading pattern. The oxychlorination reaction is exothermic. Hotspots can appear that reduce the selectivity of the reaction and give carbonisation of organic reactants inside the catalyst pellets that can break down, form fines and increase the resistance to gas flow along the tube reducing the life of the catalysts. It is important that the resistance to gas flow along each tube be about the same.

In order to reduce hotspots it has been proposed to include inert diluent beads into the reaction bed to reduce the activity of the catalysts in some part of the loading pattern. Examples include WO 2006/122 948 which describes inert diluent beads of alumina substantially of the same dimensions as the catalyst pellets. U.S. Pat. No. 4,740,644 describes diluent beads of other materials such as graphite “in a very wide variety of forms such as pellets, spheres, rings and extrudates”. A problem with existing systems is that the diluent beads, if made of the same or similar material as the catalyst pellets can be frangible, or they have poor thermal conductivity, or if made with different material they have different bulk properties and therefore do not mix well with the catalyst pellets.

U.S. Pat. No. 5,736,076 describes an oxychlorination system comprising annular catalyst pellets and beads. The catalyst pellets comprise CuCl₂/KCl/Al₂O₃. They are annular and of 5 mm diameter and length with a 2 mm bore. The diluent beads are of graphite and of similar dimensions to the pellets. The lateral compressive strength is 60 N. Additionally the bulk density of the beads is about 937 kgm⁻³, the macro porosity about 0.02 mlg⁻¹ and the BET surface area is about 5.2 m²g⁻¹. The bulk density of the catalyst pellets is not specified but will be significantly less than 937 kgm⁻³. By comparison with other known alumina pellets it is expected that the bulk density will be of the order of 760 kgm⁻³. The material of the beads has some porosity as shown by the non-negligible BET surface area. The non-negligible surface area increases the possibility of the diluent being involved in chemical transformations and especially in side reactions with the reactants decreasing yields of the desired product. The large difference in bulk density makes it difficult to achieve good mixing of the catalyst pellets and graphite beads. The result of all this is that the mixture of catalyst pellets and graphite beads is not homogeneous. The catalytic activity tends to vary between tubes and inside each tube. This results in undesirable temperature profiles and hotspots and in reduced performance and effective catalyst life.

The invention seeks to reduce this problem.

According to the invention there is provided a catalyst system for use in oxychlorination, the catalyst system comprising catalyst pellets comprising a catalyst carried on a substrate the pellets having length x, breadth y and depth z, intrinsic density P and bulk density ρ and diluent beads having length x±25%, breadth y±25% and depth z ±25%, intrinsic density>P+25% and a bulk density ρ±25%. x can be in the range 3 to 7 mm, preferably 5.5 to 6.6 mm. y can be in the range 4 to 7 mm preferably 4.5 to 5.5 mm. z can be in the range 4 to 7 mm preferably 4.5 to 5.5 mm. In some embodiments y=z±0.1 mm. The catalyst pellets can comprise alumina. The diluent beads can comprise graphite. In some embodiments the length of the beads are x±20%, preferably x±10%, more preferably x±5%. In some embodiments the breadth of the beads are y±20%, preferably y±10%, more preferably y±5%. In some embodiments the depth of the beads are z±20%, preferably z±10%, more preferably z±5%. The bulk density of the beads can be greater than ρ for example 15 to 25% greater than ρ. The intrinsic density of the beads can be 25 to 75% greater than P, preferably 50 to 65% greater than P. The thermal conductivity of the beads can be at least 5 times greater than the thermal conductivity of the pellets. The thermal conductivity of the beads can be less than 50 times greater than the thermal conductivity of the pellets. The diluent beads can have a BET surface area of less than 4 m²g⁻¹, preferably less than 1 m²g⁻¹. The pellets can be prisms or cylinders, preferably trigonal prisms or right circular cylinders. The beads can have at least one bore extending therethrough. The beads can be prisms or cylinders, preferably trigonal prisms or right circular cylinders. The beads can be right circular cylinders having a right circular cylindrical bore extending between the circular faces of the cylinder. The bore in the bead can be in the range 2.0 to 4.0 mm for example in the range of 2.2 to 2.8 mm for example in the range of 2.2 to 2.4 mm. The lateral compressive strength of the beads can be 2 to 4 times that of the pellets. In some embodiments the intrinsic density of the catalyst pellets differs from the intrinsic density of the diluent beads by at least 30% and the bulk density of the catalyst pellets differs from the bulk density of the diluent beads by no more than 15%. The invention further provides a catalyst bed for use in oxychlorination the catalyst bed comprising catalyst pellets comprising a catalyst carried on a substrate the pellets having length x, breadth y and depth z, intrinsic density P and bulk density ρ and diluent beads having length x±25%, breadth y±25% and depth z±25%, intrinsic density>P+25% and a bulk density ρ±25%. The bulk density of the catalyst bed can be constant within 5% throughout at least 75% of the depth of the bed. The invention further provides a reactor for use in oxychlorination which contains a catalyst system of the invention. The reactor can be a fixed bed reactor having a plurality of tubes each filled with the catalyst system wherein the parts by weight of the catalyst pellets of a zone of a first tube differ from the parts by weight of the catalysts pellets of a corresponding zone of a second tube by no more than 5%. The fixed bed reactor can comprise a plurality of tubes each filled with the catalyst system and which in use more than 60% of which show a pressure drop not more than 2% away from the arithmetic mean pressure drop. The invention further provides the use of the catalyst system of the invention in the preparation of 1,2-dichloroethane and in the preparation of vinyl chloride. The invention still further provides a method of preparing 1,2-dichloroethane comprising passing ethylene, hydrogen chloride and a molecular oxygen containing gas over a catalyst system of the invention. The invention yet further provides a method of preparing vinyl chloride comprising subjecting the 1,2-dichloroethane to dehydrohalogenation.

The invention further provides a catalyst system for use in oxychlorination the catalyst system comprising catalyst pellets comprising a catalyst carried on a first substrate, the pellets having length x, y and depth z and a bulk density ρ kgm-3 and diluent beads comprising a second substrate of composition different from that of the first substrate characterised in that the beads have length x±25%, breadth y±25% and depth z±25% and a bulk density as ρ±25%.

The invention further provides the use of diluent beads having each of length, breadth, depth and bulk density as independently within 25% of the corresponding parameters of catalyst pellets to control heterogeneity of a catalyst system comprising the beads and pellets.

The invention further provides a plurality of graphite diluent beads of bulk density 680-900 kgm⁻³.

The invention further provides a graphite diluent bead of length 5±2 mm, preferably 6 to 7 mm more preferably 6.2 to 6.4 mm, breadth 5.5±1.5 mm, preferably 4.5 to 6.5 mm more preferably 4.75 to 5.25 mm, depth 5.5±1.5 mm preferably 4.5 to 6.5 mm more preferably 4.75 to 5.25 mm and bore of 3±1 mm preferably 2.2 to 3.8 mm more preferably 2.2 to 2.4 mm or 2.9 to 3.1 mm.

The invention still further provides a catalyst bed comprising a mixture of catalyst pellets comprising a catalyst carried on a first substrate and diluent beads comprising a second substrate, the intrinsic density of the first substrate differing from the intrinsic density of the second substrate by at least 30% and the bulk density of the beads differing from the bulk density of the pellets by no more than 15% of the bulk density of the beads.

The invention still further provides a catalyst bed comprising catalyst pellets of a first substrate and diluent beads of a second substrate, the intrinsic density of the first substrate being different from the intrinsic density of the second substrate the bed being of substantially constant bulk density.

The invention yet further provides a catalyst bed comprising a plurality of tubes, each tube containing a mixture of catalyst pellets comprising a catalyst carried on a first substrate and diluent beads comprising a second substrate of a composition different from that of the first substrate wherein the parts by weight of the catalyst pellets of a zone of a first tube differ from the parts by weight of a corresponding zone of a second tube by no more than 5%.

Furthermore the invention provides a reactor for use in oxychlorination containing a catalyst system as set forth herein. The reactor can be a fixed bed reactor having a plurality of tubes each filled with the catalyst system wherein the parts by weight of the catalyst pellets of a zone of a first tube differ from the parts by weight of a second zone by no more than 5%. The reactor can be a fixed bed reactor for use in oxychlorination comprising a plurality of tubes each filled with the catalyst system and which in use more than 60% such as more than 65% of which such as more than 70% of which show a pressure drop not more than 2% away from the average pressure drop.

The invention further provides for the use of a catalyst system as set forth herein in the preparation of 1,2-dichloroethane or vinyl chloride.

Embodiments of the invention will be described by way of non-limiting example by reference to the accompanying figures of which

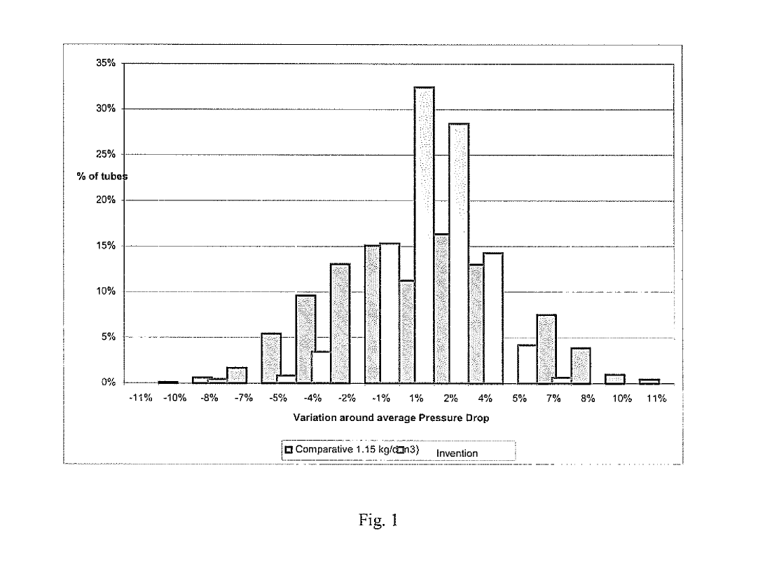

FIG. 1 is a graph showing the pressure drop across an array of tubes packed with catalyst obtained using the invention compared with a prior art array; and

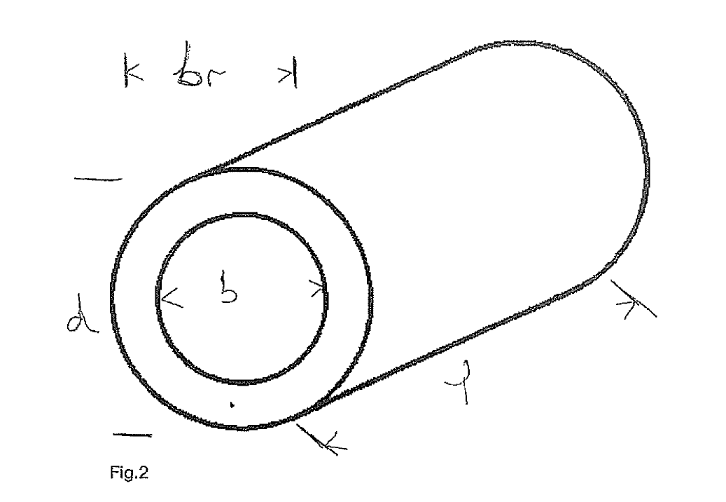

FIG. 2 is a perspective view of a diluent bead.

The external dimensions of the beads and pellets should be substantially the same. For example each of the length (as shown in FIG. 2 as “l”), depth (as shown in FIG. 2) as “d” and breadth (as shown in FIG. 2 as “br”) of the beads should independently be within 25%, for example within 20% more preferably within 15% yet more preferably within 10% still more preferably within 5% of the corresponding dimension of the pellets. The dimensions of the beads can independently be greater or smaller than the corresponding dimensions of the pellets. Preferably in order to ensure good mixing the external dimensions of the pellets and beads are at least broadly similar.

The intrinsic density of the material of the pellet and bead are different. Unless steps are taken the beads and pellets will not mix uniformly. To achieve good mixing easily the bulk density of the material of the beads should be within 25%, for example within 20% more preferably within 15% yet more preferably within 10% still more preferably within 5% of the bulk density of the material of the pellets.

Since the intrinsic density of the materials may differ by more than this amount it may be necessary to modify the pellet and/or bead. For example the bulk density of the pellet and/or bead may be reduced by forming one or more bores. Alternatively or additionally cavities either or both open or closed can be formed in the pellet or bead. Care should however be taken to ensure that the bead or pellet has sufficient crush strength to avoid significant damage during production packing and use.

Lateral compressive strength should be greater than 60 N for example 61 N or more such as 62 N or more or 100 N or more as measured by ASTM C685-91 (2010) to reduce the risk of damage or breakage during the mixing, the loading and the use of the catalyst system. Desirably lateral compressive strength of the graphite beads should be in the range of 2 to 4 times the strength of the catalyst pellets.

Bulk density could be reduced by making the bead or pellet porous for example as a sponge or sinter or by incorporating a lower density material. Bulk density could be increased by incorporating a higher density material. Those skilled in the art will have no difficulty in measuring bulk density. A method by which this can be achieved is ASTM D4164 but those skilled will be able to devise other methods.

Preferably the surface area of the diluent beads, as determined by the BET method, is kept low. A reason for this is to reduce the surface area available for competing reactions. In some embodiments therefore the BET surface area is less than 5 m²g⁻¹ preferably less than 3 m²g⁻¹ for example less than 1 m²g⁻¹. Suitable regimen determining BET surface area are ASTM D3663-03(2008) or ASTM C1274-10.

One of the functions of diluent is to conduct heat away from the catalyst. It is preferred therefore that the diluent is at least as thermally conductive as the catalyst. More preferably the diluent is at least 5 times more preferably at least 7 or 10 times as thermally conductive as the catalyst. In many embodiments of the invention the thermal conductivity of the diluent is not more than 50 or not more than 25 times the thermal conductivity of the catalyst. The precise method of determining the thermal conductivity of the materials is not of the essence of the invention provided that the same method is used for determining the conductivity of each material. Non-limiting examples of methods include ASTM E1225-09 and ASTM C177-10.

Those skilled in the art will have no difficulty in devising suitable diluents having the preferred properties. Especially where the diluent is graphite the worker of routine skill will have no difficulty in producing diluent beads of the desired properties.

It is not essential that the bulk density of the beads and the pellets be identical. While it might be thought that substantial identity will be the optimal technical solution this may not in fact be so. The intrinsic density, sometimes referred to as true density, of graphite beads is very much greater than the intrinsic density of alumina pellets. This means that in order to reduce the bulk density of the beads to that of the pellets considerable “empty volume”, defined for example by bores as shown in FIG. 2 as “b” or fins, must be present. This in turn means that sections of the pellet may be thin and prone to breakage leading to fines formation and restriction of gas flow especially where the strength of the diluent is much lower than the preferred values described herein. Alternatively or additionally it can be expensive to manufacture beads of very low density and it may be commercially desirable to provide denser beads than pellets despite somewhat less than optimal mixing. It may therefore be preferable to have the bulk density of the beads somewhat higher, for example 15 to 25% higher than the bulk density of the pellets.

The precise size and shape of the pellets and beads is not of the essence of the invention. Since surface area per unit mass is greater for small objects of the same shape than large it is desirable to make the beads and pellets small since reaction is catalysed on the pellet surface. If however the beads and pellets are too small they may pack well and thus increase unduly resistance to gas flow and the pressure drop in the reactor tubes. Each dimension of the pellets or beads may therefore be of the order of several millimetres for example 1 to 15 mm more preferably 4 to 10 mm still more preferably 6 to 8 mm.

Prisms and cylinders are easily made by extrusion and are preferable shapes. The expressions “prisms” and “cylinders” are used in the geometrical sense and are not restricted to trigonal prisms and right circular cylinders. Other shapes such as spheroids are easily made and may also be preferable.

As explained the pellets and/or beads may be provided with one or more bores or other bulk density adjusting features. Typically the beads and pellets will each have an aggregate bulk density for example as determined by ASTM D4164 in the range of about 550 to 1000 kgm⁻³ more preferably 600 to 900 kgm⁻³ yet more preferably 640 to 680 kgm⁻³ or 820 to 860 kgm⁻³. This compares with a typical bulk density of conventional cylindrical graphite beads of diameter 5.0 mm and length 6.3 mm of about 1100 to 1200 kgm⁻³ for example about 1150 kgm⁻³. Typical dimensions of the pellets are breadth 4 to 7 mm preferably 4.5 to 5.5 mm, depth 4 to 7 mm preferably 4.5 to 5.5 mm, length 3 to 7 mm preferably 5.5 to 6.6 mm with a through bore extending along the length of the pellet of 2.0 to 4.0 mm preferably 2.1 to 2.8 mm, especially 2.2 to 2.4 mm, or 2.8 to 3.2 mm. The bore or bores need not be a right circular cylinder and could for example by elliptical, star-shaped or other shapes in cross section.

The substrate of the pellet can be any of the materials known for producing copper-supported catalysts. Examples include silica, pumice, diatomaceous earth, alumina and aluminium hydroxyl compounds such as boehmite and bayerite. Preferred substrates are γ-alumina and boehmite. Boehmite may be heat treated to convert it to alumina. Typically the substrate has a BET surface area of 50-350 m²g⁻¹. The catalytically-active material supported on the substrate contains copper in an amount of 1-12 wt % based on the weight of the pellet. The copper is typically deposited on the substrate in the form of a salt especially a halide such as copper II chloride.

The copper may be used in conjunction with other metal ions for example alkali metals such as Li, Na, K, Ru or Cs, alkaline earth metals such as Mg, Ca or Ba, group IIB metals such as Zn and Cd and lanthanides such as La and Ce or mixtures thereof. These metals are typically added as salts or oxides. The total amount of additives is typically 10 wt % of metal to substrate. They can be added together with or separately (before or after or both) from the copper. Optionally heat treatment is conducted between additions. Preferred additions are Li, K, Mg, La, Cs or Ce added as chlorides in amount up to 6 wt %.

The active material and the other metal ions can be added to the substrate by for example dry impregnation, incipient wetness impregnation and dipping the substrate in an aqueous solution of the catalyst. This addition can be done before or preferably after formation of the pellet. The pellet can be subjected to thermal treatment such as calcination at 500-1100K.

The pellets and beads can be formed by for example tableting or extrusion optionally in the presence of additives such as lubricants and binders. Tableting can give more consistent sizes and stronger products than extrusion and may therefore be suitable for a wider range of shapes and density than extrusion but can be slower.

### EXAMPLES

### Calculation of Bulk Density

Bulk density can be determined by ASTM D 4164. Bulk density can also be determined by other techniques such as taking a piece of tube internal diameter 28 mm and height 470 mm. The internal volume of the tube is therefore 291.5 cm³. The beads or pellets are poured into the tube so that it is filled in 80 to 95 seconds. The beads or pellets are poured into the tube from a glass beaker of volume 1000 cm³ and initially containing 500 cm³ of pellets or beads with the lip positioned 5 cm above a funnel the tube of which has the same internal diameter of the measuring tube centered on the centre of the tube. The tube is not agitated during the determination. After the tube has been filled to overflowing the content is levelled by gently passing a straight edge across the top of the tube. It has been found experimentally that this technique gives rise to values within about 5% of that obtained by ASTM D4164.

Table 1 shows the results obtained with a range of right circular cylindrical pellets and beads using the above described, non ASTM, regimen together with other properties:

TABLE 1

| | | Difference | Difference | | | | | |
|---|---|---|---|---|---|---|---|---|
| | | in density | compared | | | | | |
| | | from | to pellet | | | | | |
| | | standard | of | BET | Thermal | | | |
| | Bulk | bead | EP 1 053 | Surface | Conductivity | Hole | External | |
| | density | diluent | 789 | area | (kcalm⁻¹h⁺¹ | diameter | diameter | Length |
| | (kgm⁻³) | (%) | (%) | (m²g⁻¹) | K⁻¹ | (mm) | (mm) | (mm) |
| Standard | 1150 | | 64 | | 90 | | 5.03 | 6.31 |
| graphite bead | | | | | | | | |
| (Comparative) | | | | | | | | |
| Hollow | 650 | −43 | −7 | 0.36 | 90 | 3.03 | 4.89 | 6.35 |
| graphite bead 1 | | | | | | | | |
| Hollow bead | 840 | −27 | 20 | 0.36 | 90 | 2.24 | 5.02 | 6.28 |
| graphite 2 | | | | | | | | |
| Pellet of EP 1 | 700 | | | | 9 | 2.25 | 4.90 | 6.35 |
| 053 789 Type A | | | | | | | | |
| Bead of U.S. Pat. No. 5,736,076 | 937 | | 26 | 5.2 | 150 | 2 | 5 | 5 |
| Ex 3 | | | | | | | | |
| (Comparative) | | | | | | | | |

It will therefore be seen that the hollow graphite beads of the invention are much closer to the bulk density of the catalyst pellets than prior art beads. It will further be seen that while the BET surface area of diluent beads is within a range routinely available, they are better i.e. lower than the values reported in U.S. Pat. No. 5,736,076.

Homogeneity testing

An industrial oxychlorination reactor was loaded in conventional manner with a mixture of pellets according to EP 1,053,789 type A and “hollow graphite beads 1” according to the invention and the pressure drop across each tube was measured. In a comparative example the same reactor was filled with a prior art mixture of standard graphite beads and pellets of EP according to EP 1,053,789 in like manner. The dimensions of the beads and pellets are given in Table 1 above.

The results are shown in the figure. It will be apparent that the variation in pressure drop across the tubes filled in accordance with the invention is much less than that obtained in the prior art. In particular it will be seen that in accordance with the invention nearly 50% of tubes were within 1% of the arithmetic mean (“average”) about 75% were within 2% of the average. Fewer than 7% of tubes were more than 5% away from the average. In the comparative example only about 33% of tubes were within 1% of the average and about 60% of tubes were within 2% of the average. Nearly 15% of tubes were more than 5% away from the average. This shows that tubes of the invention are more reproducibly packed than those of the prior art thereby reducing the likelihood of hot spots being formed.

Because the bulk density of the components of the catalyst bed are quite similar, irrespective of the loading pattern adopted, the bulk density of the catalyst bed is substantially constant and can for example differ by no more than 15%, preferably no more than 10%, still more preferably no more 5% than over at least 75% of the depth of the bed. Yet more preferably the bulk density falls within these ranges over the complete depth of the bed. In like manner the bulk density of the catalyst across the breadth of the bed is preferably substantially constant and can for example differ by no more than 15%, preferably no more than 10%, still more preferably no more 5% than over at least 75% of the breadth of the bed. Yet more preferably the bulk density falls within these ranges over the complete breadth of the bed. Where the catalyst bed comprises an array of tubes packed with catalyst and diluent the bulk density of the packed tubes is preferably substantially constant for example the bulk density of the content of at least 75% of the tubes and preferably all the tubes differs by no more than 15%, preferably no more than 10%, still more preferably no more 5% from the arithmetic mean bulk density of the tube content. Substantially constant bulk density helps assure consistency of bed packing and hence consistency of pressure drop over the bed and among the packed tubes.

1-32. (canceled)

33. A catalyst system for use in oxychlorination, said catalyst system comprising catalyst pellets comprising a catalyst carried on a first substrate, said catalyst pellets having a length x, breadth y and depth z and bulk density ρ and diluent beads comprising a second substrate different from that of the first substrate said beads having a length x±25%, breadth y±25% depth z±25% and bulk density p±25% and said diluent beads having a thermal conductivity at least 5 times greater than the thermal conductivity of said catalyst pellets.

34. A catalyst system as claimed in claim 33 wherein x is in the range 3 to 7 mm.

35. A catalyst system as claimed in claim 33 wherein y is in the range 4 to 7 mm.

36. A catalyst system as claimed in claim 33 wherein z is in the range 4 to 7 mm.

37. A catalyst system as claimed in claim 33 wherein said catalyst pellets comprise alumina.

38. A catalyst system as claimed in claim 33 wherein said diluent beads comprise graphite.

39. A catalyst system as claimed in claim 33 wherein the thermal conductivity of said diluent beads is less than 50 times greater than the thermal conductivity of said pellets.

40. A catalyst system as claimed in claim 33 wherein said pellets are trigonal prisms or right circular cylinders.

41. A catalyst system as claimed in claim 40 wherein said beads are right circular cylinders having a right circular bore having a diameter in the range 2.0 to 4.0 mm.

42. A catalyst system as claimed in claim 33 wherein the lateral compressive strength of the beads is 2 to 4 times that of the pellets.

43. A method of preparing 1,2-dichloroethane comprising passing ethylene, hydrogen chloride and a molecular oxygen containing gas over a catalyst system comprising catalyst pellets comprising a catalyst carried on a first substrate, said catalyst pellets having a length x, breadth y and depth z and bulk density ρ and diluent beads comprising a second substrate different from that of the first substrate said beads having a length x±25%, breadth y±25% depth z±25% and bulk density p±25% and said diluent beads having a thermal conductivity at least 5 times greater than the thermal conductivity of said catalyst pellets.

44. A method of preparing vinyl chloride comprising the steps of preparing 1,2-dichloroethane by a method as claimed in claim 42 and transforming it into vinyl chloride for example by cracking.

45. A reactor for use in oxychlorination containing a catalyst system as claimed in claim 33.

46. A reactor as claimed in claim 45 comprising a plurality of tubes each filled with the catalyst system wherein the parts by weight of a zone of first tube differ from the parts by weight of the catalyst pellets of a corresponding zone of a second tube by no more than 5%.
